(19) United States
(12) Patent Application Publication
(10) Pub. No.: US 2012/0247993 A1
(43) Pub. Date: Oct. 4, 2012

(54) Probiotic Compositions, Methods and Apparatus for Their Administration

(75) Inventors: Ken Palazzi, Faulconbridge (AU); Mark Styan, West Lakes (AU); Kristel Wallis, North Parramatta (AU); Jenni Sofjan, Randwick (AU); Yin Li, Strathfield (AU); Patricia Conway, La Parouse (AU)

(73) Assignee: UNISTRAW PATENT HOLDINGS LIMITED, Federal Territory of Labuan (MY)

(21) Appl. No.: 13/509,127

(22) PCT Filed: Nov. 13, 2009
(86) PCT No.: PCT/AU2009/001484

§ 371 Date: May 10, 2012

(30) Foreign Application Priority Data
Nov. 14, 2008 (AU) 2008905905
Nov. 26, 2008 (AU) 2008906124

(51) Int. Cl. A61J 7/00; A61K 9/14; A61K 9/20; A61B 19/02; A61K 36/06; A61K 36/064; A61P 1/00; B05D 7/00; A61K 9/00; A61K 35/74

(52) U.S. Cl. 206/363; 424/484; 424/464; 424/93.4; 424/93.51; 424/93.46; 424/93.45; 424/93.44; 424/485; 424/488; 427/2.14; 604/77

(57) ABSTRACT

A probiotic composition including a probiotic microorganism embedded within a matrix, the matrix substantially maintaining the viability of said microorganisms. The matrix releases said microorganisms into and upon contact with a liquid carrier. The invention includes methods for manufacturing the composition, particular forms of the composition (2) and as apparatus for administration.

### FIELD OF THE INVENTION

The present invention relates generally to probiotics and methods and apparatus for their administration.

The invention has been developed primarily for the provision of probiotic compositions that may be stored at ambient temperatures or lower while maintaining a suitable level of probiotic viability. In one form the invention has been developed to provide a probiotic composition in a format that allows for administration of the probiotic with a carrier liquid at the point of consumption. It will be appreciated, however, that the invention is not limited to this particular use, and may also be used to maintain probiotic viability in a range of applications. In addition, the invention and method may also be used in connection with other nutraceutical additives, pharmaceutical agents, dietary supplements, functional food ingredients, additives including colouring or flavouring agents, and other forms of natural or medicinal health formulations, active ingredients and supplements whether soluble or otherwise transportable in suspension.

### BACKGROUND OF THE INVENTION

The following discussion of the prior art is provided as technical background, to enable the features and benefits of the invention to be fully appreciated in an appropriate technical context. However, any reference to the prior art should not be taken as an express or implied admission that such art is widely known or forms part of common general knowledge in the field.

### Probiotics

Probiotic microorganisms are well known in the art and the term refers to live microbial preparations that may be administered to a subject in order to confer a beneficial effect, such as restoring or improving the composition of intestinal microflora. Probiotics are typically provided as dietary supplements containing potentially beneficial bacteria or yeast and are widely consumed in foods as well as in capsules and powders (Stanton et al, Market potential of probiotics. Am J Clin Nutr 73 (suppl):476S-83S.). Generally, lactic acid bacteria including Lactobacillus and Bifidobacterium are used as probiotics but other genera are also used including Lactococcus, Propionibacterium, Bacillus, Saccharomyces as well as strains of Escherichia. Within these genera, many species and strains have been reported to have probiotic properties. The most common vehicles for the delivery of probiotics are dairy products and probiotic fortified foods. However, powders, tablets and capsules containing probiotics are also available.

The different strains of bacteria and yeast vary in the probiotic benefits they can to provide. However, a common problem for all probiotics is that of survival during processing and storage since it is well reported that the microbes must be alive to be active and confer the maximum beneficial effect.

### Maintaining Probiotic Survival

Bulk probiotics are generally supplied as powders prepared by adding cryoprotectants to concentrates from fermentation vessels prior to freeze drying. However, spray drying of concentrates is also used. A range of additives have been used as cryoprotectants to preserve viability of the probiotic during production and storage, e.g. US 20050100559. A variety of additives have also been reported in scientific publications and in patent applications. Microencapsulation processes are reported using a range of coating ingredients often in combination with spray drying (e.g. US20070122397, US20070059296, and US20040223956). Freeze drying is carried out either directly after harvesting or by freezing droplets in liquid nitrogen and then drying the frozen droplets. The resultant dried material is then milled to form a powder. This powder and finished products are typically stored chilled as ambient temperature often results in poor survival. Considerable research has targeted improving survival by addition of additives during post processes and also during fermentation (e.g. U.S. Pat. No. 6,939,705). The survival of probiotics in soft chewable confectionary has also been reported (US 20060263344). A desirable probiotic composition would be one that can maintain a beneficial amount of viable microorganisms during storage at ambient temperatures. Further, a method and apparatus for administering probiotic compositions to children and infants would be desirable.

It is an object of the present invention to overcome or substantially ameliorate one or more of the limitations of the prior art, or at least to provide a useful alternative.

### SUMMARY OF THE INVENTION

According to a first aspect there is provided a probiotic composition comprising a probiotic microorganism embedded within a matrix, said matrix substantially maintaining the viability of said microorganisms, whereby said matrix releases said microorganisms into and upon contact with a liquid carrier.

Preferably, the matrix is substantially free of water and may be dried by methods known in the art such as freeze-drying, spray drying, fluid bed drying, pan drying, tray drying, oven drying, vacuum drying or lyophilisation. In order to improve or maintain probiotic microorganism viability, the matrix preferably comprises one or more materials that substantially maintain the viability of the probiotic microorganisms during storage at ambient temperatures.

The matrix may include aqueous or non-aqueous components. In a preferred embodiment the matrix includes one or more components selected from the group consisting of various starches, maltodextrin, sugars, proteins, edible oils, fats, fatty acids, silicon dioxide, gums, milk compounds or derivatives, binding agents and emulsifiers. It will be understood that some of these components may also act as pre-biotics, encouraging survival, growth and proliferation of the probiotic microorganisms upon consumption. Non-aqueous matrix components other than well known edible oils and fats may be selected from various edible and/or pharmaceutical grade polyols. The matrix may additionally incorporate known disintegrants, “push” compounds and the like, to assist with release of the probiotic microorganisms from the matrix.

Preferably, the probiotic bacteria are selected from, but not limited to, the group comprising Lactobacillus, Bifidobacterium, Lactococcus, Propionibacterium, Bacillus, Enterococcus, Streptococcus and Escherichia. However, non-bacterial probiotics including probiotic yeasts such as Saccharomyces are also known and contemplated for use in the present invention. In a preferred embodiment the probiotic microorganisms are selected from Lactobacillus acidophilus, Lactobacillus rhamnosus, Lactobacillus fermentum, Lactobacillus casei, Lactobacillus bulgaricus, Lactobacillus gasseri, Lactobacillus helveticus, Lactobacillus johnsonii, Lactobacillus lactis, Lactobacillus plantarum, Lactobacillus reuteri, Lactobacillus salivarius, Lactobacillus paracasei, Bifidobacterium sp, Bifidobacterium longum, Bifidobacterium infantis, Bifidobacterium animalis, Bifidobacterium bifidum, Bifidobacterium adelocentis, Bifidobacterium lactis, Enterococcus faecalis, Enterococcus faecium, Lactococcus lactis, Streptococcus salivarius, Saccharomyces cereviscae and Saccharomyces boulardii.

It will be appreciated however that the above list of probiotic microorganisms is non-limiting, and the skilled addressee would understand that a number of other commercially available probiotic microorganisms can be used in the compositions of the present invention.

In a preferred embodiment the probiotic microorganisms are selected from genera Lactobacillus, Streptococcus and Bifidobacterium. In a most preferred embodiment the probiotic microorganisms are selected from the group consisting of Lactobacillus acidophilus Lactobacillus rhamnosus, Lactobacillus fermentum, Lactobacillus plantarum, Streptococcus salivarius, Bifidobacterium lactis and Bifidobacterium infantis. In some embodiments, the compositions of the invention comprise probiotic microorganisms of two or more genera, species or strains and/or genetically modified microorganisms. In this regard, the term “microorganism” or “probiotic” is intended to include reference to a single genus, species and/or strain or mixtures of genera, species and/or strains and is also intended to refer to genetically modified microorganisms, which may confer alternative or additional benefits. Further, in the context of the present invention the terms “probiotic” and “probiotic microorganism” may be used interchangeably.

The probiotic microorganism may be released from the matrix by the mechanical effect of the liquid carrier, where the liquid carrier moves over and/or through the matrix and also as the matrix wholly or partially dissolves, disintegrates and/or erodes into the liquid carrier.

Thus, the matrix may be totally or partially soluble in the liquid carrier or include ingredients that are totally or partially soluble in the liquid carrier. Either way, the terms “dissolved”, “dissolvable” and the like, as used herein, are intended to be construed sufficiently broadly to encompass not only dissolution in the strict chemical sense, but also suspensions, slurries and mixtures formed with the carrier liquid. Accordingly the term “soluble” when applied to substances and materials refers to the property of such materials and substances to be dissolved or dissolvable as herein defined. Thus, in the context of the present invention the terms “soluble”, “dissolvable” or “dissolved” are intended to encompass matrices which may fully dissolve or solubilise in a carrier fluid as well as matrices which merely disintegrate or erode in contact with a carrier fluid

Preferably, the liquid carrier is a moving fluid and the term “liquid carrier” refers to any liquid suitable for ingestion and includes pharmaceutical formulations and foodstuffs such as water, milk, fruit juices, vegetable juices, electrolytic beverages and the like.

The compositions of the invention may also include other additives including flavourings, colourings, nutrients, supplements, excipients, vitamins, recombinant products, and other useful or beneficial additives as are known in the art. An extensive list of additives, particularly food additives and their known usage can be found at, for example, http://www.nutritiondata.com/topics/food-additives.

Further, the compositions may include one or more pharmaceutically active agents, such as antibiotics, analgesics and the like. The pharmaceutically active ingredient may be mixed with pharmaceutically acceptable carriers/excipients such as inert diluents, disintegrating agents, binding agents, lubricating agents, sweetening agents, flavouring agents, colouring agents and preservatives. Examples include, but are not limited to, any of the standard pharmaceutical carriers such as a phosphate buffered saline solution, water, emulsions such as oil/water emulsion, and various types of wetting agents. Examples of other excipients include but are not limited to magnesium stearate, lactose, microcrystalline cellulose, stearic acid, gelatin, sucrose and acacia. For methods of preparing various types of formulations and choice of carriers, excipients and additives, see standard pharmacy texts and manuals, such as for example, Remington: The Science and Practice of Pharmacy, 19^{th} Ed., Mack Publishing Co., 1995, the Theory and Practice of Industrial Pharmacy, Lachman L., et al. Lea & Febiger, Philadelphia 3^{rd} Edition, Bentley's Textbook of Pharmaceutics Ed. E A Rawlings Ballilliere Tindall, London 8^{th} Edition, incorporated in its entirety herein by reference.

According to a second aspect there is provided a probiotic composition in pellet form comprising a probiotic microorganism embedded within a matrix, said matrix substantially maintaining the viability of said microorganisms, and whereby said matrix releases said microorganisms into and upon contact with a liquid carrier.

According to a third aspect there is provided a probiotic composition in pellet form comprising a core bead and a probiotic microorganism embedded within a matrix, said matrix substantially maintaining the viability of said microorganisms, wherein the matrix is disposed on or in the core bead and whereby said matrix releases said microorganisms into and upon contact with a liquid carrier.

The pellet-form of the composition facilitates rapid dissolution, disintegration or erosion of the matrix and release of the probiotic microorganisms from within the matrix into the liquid carrier.

Preferably the core bead is porous or semi-porous so that the matrix containing the probiotic microorganism can be impregnated into the bead core to form a porous pellet or bead. Such a porous pellet may be covered by one or more layers depending on requirements (eg. protection of the pellet and/or microorganism, addition of flavours and colour, addition of pre-biotics or different probiotic microorganisms, addition of nutritional components and the like).

The core bead and/or the matrix may be soluble in a liquid carrier, thus releasing the probiotic microorganism. However it will be understood that the probiotic microorganism may be released just as effectively by disintegration or erosion of the bead core and/or the matrix.

The preferred matrix is a non-aqueous matrix comprising an oil. Choice of suitable oils and other non-aqueous matrix components is discussed in more detail below.

According to a fourth aspect there is provided a probiotic composition in pellet form comprising a core bead and a probiotic microorganism embedded within a matrix, said matrix substantially maintaining the viability of said microorganisms, wherein the matrix is disposed on the core bead in plurality of layers to form a layered pellet and whereby said matrix releases said microorganisms into and upon contact with a liquid carrier.

The term “pellet” as used herein refers to discrete units and includes such things as beads that are generally spherical in shape but also encompasses different shapes including prolate spheroids, oblate spheroids, cylinders, rods, prisms or other regular geometric, or irregular shapes. The term “pellet” may be used interchangeably with the term “bead”. The term “bead core” as used herein may be used interchangeably with the term “core” and is intended to describe an entity such as a sugar crystal or the like, commonly used as a starting point in preparation of beads or pellets. The term “core bead” is used herein to describe the bead or pellet, into or onto which the probiotic preparations are applied.

More preferably, the probiotic composition is in the form of a substantially spherical bead. Substantially spherical beads of a similar size or sized within a defined range provide a more consistent dissolution profile allowing for an approximate dosage to be delivered in an approximate time.

In one embodiment, the pellet or bead is substantially homogeneous in composition and the microorganism is located throughout the bead or pellet. Thus, the bead or pellet may be porous or semi-porous, allowing the matrix components and probiotic microorganisms to impregnate the spaces within the bead or pellet. Preferred compounds include pectins, gelatins, hydrocolloid gels and other like substances. The probiotic microorganisms may be released from such a pellet or bead by percolation of carrier liquid through the bead or pellet, or by dissolution, disintegration or erosion of the porous bead or pellet. In addition the porous bead or pellet may be coated with additional layers that serve as a protective coat or alternatively include matrix components as discussed above.

In some embodiments, the pellets or beads are formed as “nonpareil” pellets, which is a term of art in food and confectionary manufacturing to indicate a structure formed by building up successive layers of material. As used herein, the term is intended to embrace a similar meaning in terms of the accumulation of successive layers formed from probiotic components, matrix, flavouring, nutritional or pharmaceutical formulations, in the context of the present invention.

Since each pellet can be formed layer by layer, the composition of each layer may be varied as required. Advantageously, this enables the dissolution, disintegration or erosion characteristics of the pellets to be controlled so that as they are progressively reduced in size during the dissolution process, the shape of each pellet or bead tends to be substantially retained.

Furthermore, a layered construction allows control over the distribution of additives, which may include one or more probiotic microorganisms, in the pellets. For instance, the probiotic microorganism may be located in any one or more layers of the bead. Different combinations of probiotic microorganisms and other additives can be distributed throughout the layers.

As such the layered construction may provide control over the order the probiotic microorganisms and other additives are released into the liquid carrier. For instance, by placing a pre-biotic in an outer layer of the pellet and a pro-biotic in an inner layer of the pellet, the pre-biotic can be released into the carrier liquid generally in advance of the probiotic microorganism.

In another embodiment, the matrix is applied in successive layers, one or more of which need not be soluble in the carrier fluid. Preferably the one or more layers includes an oil, fat, fatty acid, shellac or wax. More preferably the oil, fat, fatty acid, shellac or wax is an edible oil, fat, fatty acid, shellac or wax. Preferably the successive layers contain different microorganisms.

A particular process can involve coating beads using a non-aqueous matrix such as an oil, in a panning process that involves successive additions of tapioca starch-maltodextrin powder and oil alone or with added gum acacia (2.5%) and/or added silicon dioxide. Preferably, but not essential, the starting material, i.e. bead, is super-dried (water activity of preferably no more than 0.1). Preferably the oil is a plant/vegetable oil. More preferably the oil is canola.

Some of the many different kinds of suitable/acceptable oils include but are not limited to olive oil, palm oil, soybean oil, canola oil, pumpkin seed oil, corn oil, sunflower oil, safflower oil, peanut oil, grape seed oil, sesame oil, argan oil, coconut oil and rice bran oil.

This preferred embodiment can also be prepared using other food grade acceptable or edible oils or coatings as described in The Handbook of Australasian Edible Oils (Publisher—Oils and Fats Specialist Group NZIC) or the Handbook of Food Preservation (edited by M. Shafiur Rahman, CRC Press—and which can be found at:

http://books.google.com/books?id=sKgtq62GB_gC&pg=PA482&lpg=
PA482&dq=EDIBLE+OILS+COATINGS&source=web&ots=
DEJ62MNDHQ&sig=sJP9yjJXG6qp2uv7KS424OoV4Ks&hl=en&sa=
X&oi=book_result&resnum=8&ct=result#PPP1,M1.

Alternative non-aqueous components may be selected from food or pharmaceutical grade polyols and the like such as propylene glycol, other glycol compounds, sterols etc.

Preferably, the liquid carrier is a moving fluid which moves from a source, such as a container, into contact with the matrix comprising the probiotic microorganism such that the matrix is solubilised and the microorganism is released, whereby the flow of liquid then carries the microorganism to the subject for ingestion. Still more preferably, the pellets or beads are adapted to dissolve within a standard volume of about 200 mL when the probiotic microorganism is dispersed throughout the pellet or bead or in both inner and outer layers of the pellet or bead. Of course, the required volume to release and administer the probiotics would be less where the probiotic microorganism is located only in an outer layer of the pellet or bead such as at the surface or where the matrix is designed to dissolve more rapidly.

According to a fifth aspect, there is provided a method of producing a layered pellet comprising a probiotic microorganism said method comprising:

i) providing a bead core;

ii) adhering a layer of matrix material to said core to provide a layered pellet; and

iii) optionally further contacting said layered pellet one or more times with the matrix material to apply one or more additional layers,

wherein at least one of steps i, ii or iii includes matrix material comprising a probiotic microorganism, such that the layered pellet includes a probiotic microorganism.

Preferably the bead core is a soluble sugar crystal. Suitable sugars include glucose, fructose, sucrose, lactose, trehalose, maltose and other suitable sugars as are known in the art. It will be understood however that neither the bead core nor the layered material need be soluble in order to be able to release the probiotic microorganism when contacted with a carrier liquid. Thus, the bead core and/or the layered material may disintegrate or erode when exposed to the carrier liquid.

The sugar core could also be a low energy sugar substitute such as sorbitol, manitol, xylitol or other sugar substitute known in the art.

In one or more embodiments the location of the probiotic microorganism in or on the pellet or bead is selected from an outer layer, an inner layer a mid layer, the core or any combination of outer, inner and mid layers or core of the pellet or bead.

Preferably one or more of the layers of the pellet includes an oil. More preferably the oil is an edible oil. The successive layers may contain different microorganisms.

In another embodiment the bead core may be porous, as described earlier, enabling the bead core to be impregnated with the probiotic microorgansim, and may additionally have a surface protective coating layer, or one or more layers that may include probiotic bacteria or other ingredients such as flavouring, colouring and the like. Further, the bead core may be prepared by an extrusion, granulation or other process.

Thus, according to a sixth aspect, there is provided a method of producing a probiotic composition in pellet form, said method comprising:

i) providing a porous or semi-porous bead core;

ii) impregnating said core with a matrix comprising a probiotic microorganism; and

iii) optionally applying one or more surface coating layers on said impregnated core.

According to a seventh aspect, there is provided a pellet composition comprising a probiotic microorganism when produced by a method according to the invention.

Preferably, the layered pellets according to the invention are soluble and comprise a matrix or material that includes one or more components selected from the group consisting of various starches, maltodextrins, sugars, proteins, edible oils, fats, fatty acids, silicon dioxide, gums, milk compounds and their derivatives, hydrocolloids, binding agents and emulsifiers. It will be understood that some of these components may also act as pre-biotics, encouraging survival, growth and proliferation of the probiotic microorganisms upon consumption. Non-aqueous matrix components other than oils and fats may be selected from various edible and/or pharmaceutical grade polyols. The matrix may additionally incorporate known disintegrants, “push” compounds and the like, to assist with release of the probiotic microorganisms from the matrix.

In embodiments that make use of the porous or semi-porous bead core, the core and/or the matrix employed for impregnating the core with the probiotic microorganism may be soluble or alternatively may disintegrate or erode on contact with the carrier liquid. An advantage of a porous bead core is that a greater number of viable probiotic microorganisms may be introduced within each pellet and thus allows greater doses of viable probiotic microorganisms to be delivered.

Preferably, one or more different probiotic compositions, comprising a different probiotic microorganism can be mixed together. Each of the compositions may include different components, including different type of pellet configuration and content.

According to another aspect there is provided a container containing a probiotic composition wherein said container is hermetically sealed.
Preferably, said container is a disposable sachet including a measure of probiotic composition having a predetermined dose of probiotic.

In another embodiment, there is provided a probiotic composition according to the invention for use in an elongated tube, drinking straw or the like.

According to another aspect, there is provided a receptacle containing a plurality of pellets or beads comprising a probiotic matrix composition for dispersion into a carrier liquid according to the invention, said receptacle including a body adapted to contain said plurality of pellets or beads, and a pair of filters disposed in spaced apart relationship and adapted substantially to retain said plurality of pellets or beads within the body while permitting relatively passage of the carrier liquid therethrough.

In one form the receptacle is part of, or adapted for connection to an elongate tube adapted for use as a straw, thereby enabling the carrier liquid to be drawn through the receptacle and the probiotic microorganisms to be dispersed into the carrier liquid within the tube.

In some embodiments, the filters preferably include perforations being sufficiently small in size to retain the first pellets or beads within the receptacle and to retain the second pellets or beads within the straw until they have substantially dissolved, and being sufficiently large in size to permit relatively unimpeded flow of the carrier liquid through the straw under moderate levels of oral suction.

Moreover, the term “pair” as used in connection with filters is not intended to imply that such filters are necessarily identical or even similar in form or structure to one another, or to other filters that may be used in combination therewith.

In some embodiments, the filters are disposed at, on or adjacent opposite ends of the body. In other embodiments, however, one or both of the filters may be disposed at intermediate positions within or along the length of the body.

In some embodiments, the receptacle is formed as an integral part of, or is defined by, the tube that forms the straw. For example, in one embodiment, the receptacle is defined as a chamber, compartment or region within the straw itself, noting that the chamber may be defined in part by one or more filters and/or valves. In other embodiments, however, the receptacle is formed as a separate and discrete component, adapted for connection to or integration with the straw as part of a subsequent process step, manufacturing operation or assembly procedure.

In some embodiments, the pellets or beads are generally spherical in shape, having an average diameter that is between 5% and 95% and preferably between 10% and 90%, of the internal diameter of the body. In other embodiments, the generally spherical pellets or beads are between 20% and 80%, and in other embodiments between 25% and around 75%, of the internal diameter of the body. The body itself, in some embodiments has an internal diameter of between 3 mm and 15 mm, and in some embodiments between 7 mm and 9 mm. In some embodiments, the pellets or beads have an average diameter of between 1 mm and 8 mm, and ideally between 1.5 mm and 3 mm. In cases of non-circular straws and/or bodies, or straws and or bodies of non-uniform cross section, it will be appreciated that the above dimensions form a rough guide. For instance, it is preferable that the maximum and minimum widths of such straws and/or bodies fall within the nominated 3 mm to 15 mm range and preferably within 5 mm to 12 mm.

In one particularly preferred embodiment the internal diameter of the straw is around 8 mm and the pellets are between 1 mm and 3 mm. In another particularly preferred embodiment the internal diameter of the straw is around 5 mm and the pellets are between 1 mm and 3 mm. In some embodiments, the innermost layers of at least some of the pellets or beads contain probiotic microorganisms and/or additives at concentrations greater than those in the outermost layers. Advantageously, this increase in concentration offsets the reduction in surface area of the pellets or beads as they progressively dissolve, thereby imparting a relatively uniform concentration of a probiotic microorganism and flavourings or other ingredients during consumption.

The probiotic beads or pellets can be used with an additional ingredient or ingredients within the straw, receptacle or tube whereby said probiotic microorganisms and said additional ingredient or ingredients are progressively dispersed into the carrier liquid within the straw, receptacle or tube. Additional ingredients can include pharmaceuticals, vitamins, minerals, nutritional supplements, health tonics, colouring agents or flavouring agents may be used

In some embodiments, the concentration and/or dissolution rate of the probiotics and optional additional ingredient(s)/component(s) are selected such that substantially all of them are dissolved and delivered upon consumption of a predetermined volume of an intended carrier liquid. In one such embodiment, the straw is packaged and sold in combination with a complementary container, which includes or is adapted to contain the predetermined volume of the intended carrier liquid.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE DRAWINGS

Preferred embodiments of the invention will now be described, by way of example only, with reference to the accompanying drawings in which:

FIG. 1 shows a cross-sectional view of the pellet or bead with the probiotic microorganism in the outer layer;

FIG. 2 shows a cross-sectional view of the pellet or bead with multiple probiotic microorganism and or additives in layers separated by a barrier layer;

FIG. 3 shows a cross-sectional view of the pellet or bead with the probiotic microorganism in multiple layers with an outer protective barrier layer;

FIG. 4 shows a cross-sectional view of the pellet or bead having a porous or semi-porous bead core impregnated with a probiotic microorganism and an outer protective layer;

FIG. 5 is a perspective view of drinking straw containing a first active ingredient, according to the invention;

FIG. 6 is a plan view of the of the straw shown in FIG. 1;

FIG. 7 is a plan view of an alternative embodiment drinking straw including first and second chambers containing first and second pellets having first and second active ingredients respectively, according to the invention;

FIG. 8 is a plan view of an alternative embodiment drinking straw including multiple chambers containing multiple pellets having multiple active ingredients respectively, according to the invention.

### PREFERRED EMBODIMENTS OF THE INVENTION

The invention provides a probiotic composition including a probiotic microorganism for administering to a mammal via a carrier liquid and extends to a method and apparatus for administering the composition into the carrier liquid.

The probiotic composition includes probiotic microorganisms embedded within a soluble matrix. The probiotic microorganisms are typically probiotic bacterial microorganisms, although other probiotics microorganisms as are known in the art may also be used, for example yeast such as Saccharomyces.

The bacterial genera may be selected from any commonly used or known probiotic microorganisms such as, for example, Lactobacillus, Bifidobacterium, Lactococcus, Propionibacterium, Bacillus, Enterococcus, Escherichia, and Saccharomyces and in the most preferred embodiments, these bacteria are selected from the group comprising Lactobacillus acidophilus, Lactobacillus rhamnosus, Lactobacillus fermentum, Lactobacillus casei, Lactobacillus bulgaricus, Lactobacillus gasseri, Lactobacillus helveticus, Lactobacillus johnsonii, Lactobacillus lactis, Lactobacillus plantarum, Lactobacillus reuteri, Lactobacillus salivarius, Bifidobacterium sp, Bifidobacterium longum, Bifidobacterium infantis, Bifidobacterium animalis, Bifidobacterium bifidum, Bifidobacterium adelocentis, Enterococcus faecalis, Enterococcus faecium, Lactococcus lactis, Saccharomyces cereviscae, Saccharomyces boulardii and Lactobacillus paracasei. It will be appreciated by one skilled in the art that appropriate probiotic microorganisms may be selected from those known in the art and that mixtures of probiotic microorganisms may be used to suit particular applications, for example when it is desired to provide multiple or complimentary health benefits.

The soluble matrix, within which the probiotic microorganisms are embedded, acts to substantially maintain the viability of the microorganisms during storage and is dissolvable or dispersible on contact with a liquid carrier to release the microorganisms into the liquid carrier. The matrix components must be transportable in a liquid carrier, either in dissolved or particulate form but it is preferable that the matrix components be soluble. One or more different probiotic compositions having a similar or different matrix, comprising a different probiotic microorganism can be mixed together. This allows delivery of one or more microorganisms, each having an optimal survival rate.

The soluble matrix may also include one or more components selected from the group consisting of carbohydrates, milk products, binding agents, thickening agents, emulsifiers, oils, fats, fatty acids, waxes, water, silicon dioxide and proteins and other food-grade components as are known in the art. More particularly, the components are selected from the group consisting of resistant starches, dextrins, sugars, skim milk, gelatin, canola oil, water, silicon dioxide, sucralose, gum acacia, soy proteins and lecithin. In a preferred embodiment, the matrix includes one or more components selected from the group consisting of resistant tapioca starch, maltodextrin, lactose, trehalose, skim milk powder, gelatin powder, water, silicon dioxide, sucralose, gum acacia, soy protein, and lecithin. It will be appreciated by one skilled in the art that other components as are known in the art may be also selected to suit particular applications.

It will be appreciated however that the identity of the probiotic bacteria to be used is not critical and the above list of probiotic microorganisms is non-limiting. The skilled addressee would understand that a number of other commercially available probiotic microorganisms can be used.

It should also be appreciated, that in addition to a probiotic, other active ingredients may be included in the soluble matrix. For instance, other active ingredients that may be included along with the probiotic microorganism include, pharmaceuticals, vitamins, minerals, nutritional supplements, health tonics, energy supplements, stimulants, colouring and/or flavouring agents.

In other forms of the invention, the pellets, including the probiotic microorganisms and matrix components may be provided in a variety of suitable forms depending on the required application and methods of storage and subsequent delivery. Accordingly, the pellets may be replaced by particulates, powders, tablets in other forms such as ribbons, blocks, and non-concentric shapes such as cubes, polygons and the like or concentric shapes such as spheres, cylinders/rods, discs and the like. For example, a composition according to the invention could be provided in a relatively large block and subsequently milled prior to administration or pelleting or extruded and re-packaged prior to administration.

Advantageously, however, the compositions are provided in a format that can be readily dissolved in a liquid carrier for rapid administration such as in particulate, granular or pellet form. In this embodiment, the composition is provided in the form of substantially spherical beads to facilitate passage of the carrier liquid past entrapped beads. The use of pellets or beads is also advantageous in order that the compositions remain entrapped within the filter receptacle.

The probiotic composition is in the form of layered or “nonpareil” pellets or beads. “Nonpareil” beads comprise successive layers of material. These beads include layers that comprise probiotic microorganisms and, optionally, other layers that do not contain probiotic microorganisms.

Advantageously, a layered construction enables the dissolution characteristics of the pellets to be controlled, and facilitates shape retention as the pellets progressively diminish in size during the dissolution process. In addition, the layered construction enables the composition or concentration of each layer to be varied as required.

The use of nonpareil pellets or beads allows the composition of the pellet or bead to be varied between different layers. The nonpareil beads are produced by building up layers of soluble material on a bead core or seed. In this embodiment, the bead core is a sugar crystal, although other soluble materials may be used as are known in the art.

In other embodiments, the bead core may be insoluble. The insoluble bead core would be large enough to prevent them from passing through either filter such that only the soluble exterior layers or coatings applied to the surface of the insoluble bead core would be solubilised and pass though the filer for oral consumption.

The layers can be added by a process known as “panning” whereby successive alternating layers of adhesive syrup or binding agent and a soluble matrix powder are built up on the bead core. In at least some of the layers, the powder or the syrup or binding agent, includes the probiotic composition.

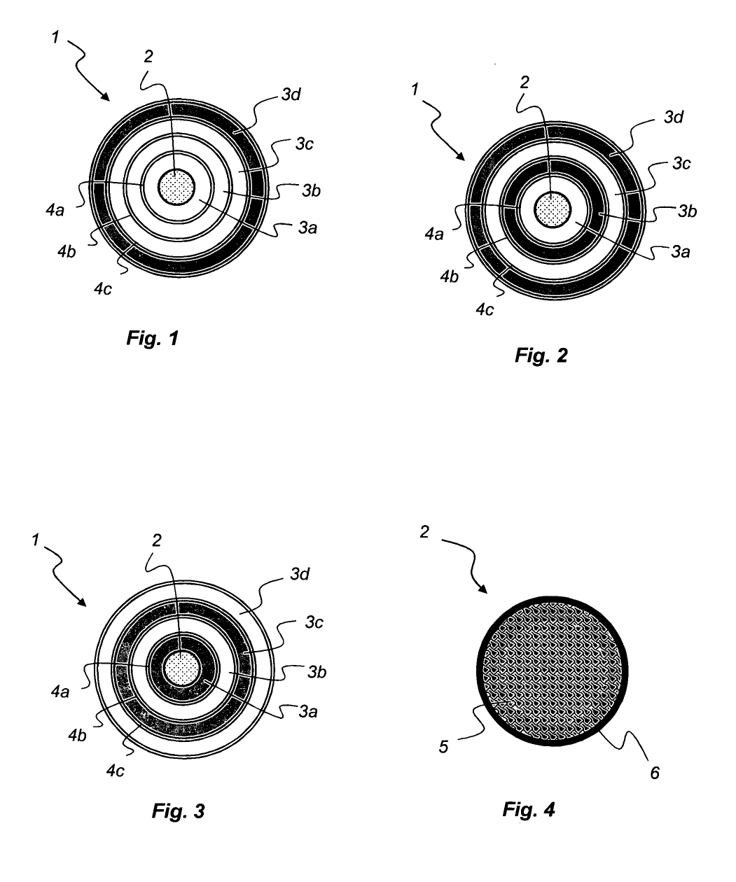

FIGS. 1 to 3 show schematic representations of a nonpareil pellet 1 in accordance with the invention, each pellet having a core, 2 and number of matrix layers 3a, 3b, 3c and 3d. It will be appreciated that while these figures display only four layers, many more layers may be present in the actual pellet. Syrup layers 4a, 4b and 4c are shown between each of the matrix layers. In the figures, the presence of the microorganism is indicated by the respective layer being shaded.

FIG. 1 shows a pellet having a probiotic composition in the outer matrix layer 3d. The inner layers of this pellet are comprised of matrix and syrup that do not contain the microorganisms. Alternatively, the microorganism may be located in one or more layers 3b and 3d of said pellet or bead as shown in FIG. 2. The microorganism and/or additives may also be different in each of these two layers and in this case they are separated by a spacing or barrier layer, 3c. A similar concept is applied to the bead shown in FIG. 3 however in this case the outer layers 3d do not include the probiotic and thereby provide a protective coating over the microorganism layers 3a and 3c below.

The process for production of the layered beads is conducted at ambient temperature or above, especially 30-33° C. However, the person skilled in the art will appreciate that the temperature may be varied in accordance with the equipment used, the relevant materials used and the relative heat sensitivity of the probiotic microorganisms involved.

In one embodiment, the beads are grown from the core (seed) by panning the bead core with 1-6 successive alternating layers of adhesive syrup and tapioca starch-maltodextrin powder containing the probiotic. Subsequent layers were then added using sugar syrup and starch-maltodextrin powder without probiotic to provide a soluble protective coating over the inner layers. The illustrated beads are approximately 2 mm in diameter and contain approximately 10⁶ to 10¹⁰ viable microbial cells per gram. Of course, the size of the beads, the number of layers included in the beads and the number of viable cells may be varied for particular applications.

In the preferred embodiments these probiotic bacteria are selected from, but not limited to, Lactobacillus acidophilus, Lactobacillus rhamnosus, Lactobacillus fermentum, Lactobacillus casei, Lactobacillus bulgaricus, Lactobacillus gasseri, Lactobacillus helveticus, Lactobacillus johnsonii, Lactobacillus lactis, Lactobacillus plantarum, Lactobacillus reuteri, Lactobacillus salivarius, Lactobacillus paracasei, Bifidobacterium sp, Bifidobacterium longum, Bifidobacterium infantis, Bifidobacterium animalis, Bifidobacterium bifidum, Bifidobacterium adelocentis, Enterococcus faecalis, Enterococcus faecium, Lactococcus lactis, Saccharomyces cereviscae and Saccharomyces boulardii.

It will be appreciated however that the identity of the probiotic bacteria to be used is not critical and the above list of probiotic microorganisms is non-limiting. The skilled addressee would understand that a number of other commercially available probiotic microorganisms can be used.

In a preferred formulation the starch-maltodextrin powder comprises 80 g of tapioca starch, 20 g of maltodextrin 2.5 g of silica dioxide and 1 g of sucralose as an optional ingredient. The adhesive syrup comprises 2.5% gum acacia, 5% soy protein, 2% canola oil, 0.25% lecithin, 10% lactose, 30% maltodextrin and 50% water. However, the amount of the various components in the powder and syrup mixtures may be altered to suit the particular application. The outer layers of the beads are comprised of alternating layers of adhesive syrup and starch-maltodextrin powder as shown above but without probiotic. When aqueous based agents are used in the bead or pellet production, final beads need to be dried to reduce the water activity, preferably at or below 0.1. Drying can occur using freeze drying, fluid bed drying, pan or tray drying or other drying method as may be applicable and suitable.

After drying of the beads to achieve a suitably low water activity, the beads are added to an apparatus for administering the probiotic composition as described above.

As discussed, the layered construction allows the characteristics of the pellets to be tailored as required. For instance the inner layers of at least some of the pellets can contain ingredients at concentrations greater than those in the outer layers.

Advantageously, this increase in concentration offsets the reduction in combined surface area as the pellets are progressively dissolved, thereby tending to maintain a relatively uniform concentration of ingredients during consumption. In terms of flavouring agents, this can be important because of the desirability for the user to experience a relatively uniform, sustained flavour as the beverage is progressively consumed, so as to simulate the taste experience of a uniformly pre-mixed beverage. In terms of probiotics and some pharmaceuticals, this functionality can be important because of the desirability of relatively uniform ingestion from a medicinal perspective.

In one variation, the probiotic is confined to the outer layers of the associated pellets, while a colouring and/or flavouring agent is used to indicate that layer has dissolved. In this way, once the active ingredients have been dissolved, the colour and/or flavour of the carrier liquid markedly changes, to provide a clear visual indication that the prescribed dosage has been fully dispensed and/or a pleasant or unique aftertaste, following consumption of the medication. Such a colour and/or flavour change can be used to both alert the user that the medication has been consumed, and/or provide encouragement and incentive to achieve full consumption and correct dosage.

In another variation, schematically depicted in FIG. 4, a porous or semi-porous bead core 2 is impregnated with a probiotic microorganism 5 combined with a suitable aqueous or non-aqueous matrix, to form a porous bead. Additional layers 6 may be optionally applied to such an impregnated porous bead, either to protect the surface of the bead core and/or the probiotic microorganism, or to include other desired ingredients (eg. flavours, colour, pre-biotics, additional probiotic microorganisms of same or different variety, nutritional components and the like) in one or more external layers.

It will be appreciate by one skilled in the art that the above-described layered beads or porous beads may be altered and adapted to suit particular applications. For instance, in one embodiment the probiotic compositions of the invention may be provided in a sealed container such that a measure of probiotic pellets or beads could be removed from the container and mixed with and dissolved into a carrier liquid prior to consumption. Preferably the container is hermetically sealable to substantially prevent ingress of external elements such as water, air and water vapour.

In another particular form, the container is a hermetically sealed disposable sachet manufactured at least in part from a vapour proof material. The sachet includes a measure of the probiotic composition sufficient to provide a predetermined, individual dosage. The dosage may be determined to be sufficient to be taken at set intervals, for instance, hourly, daily or weekly.

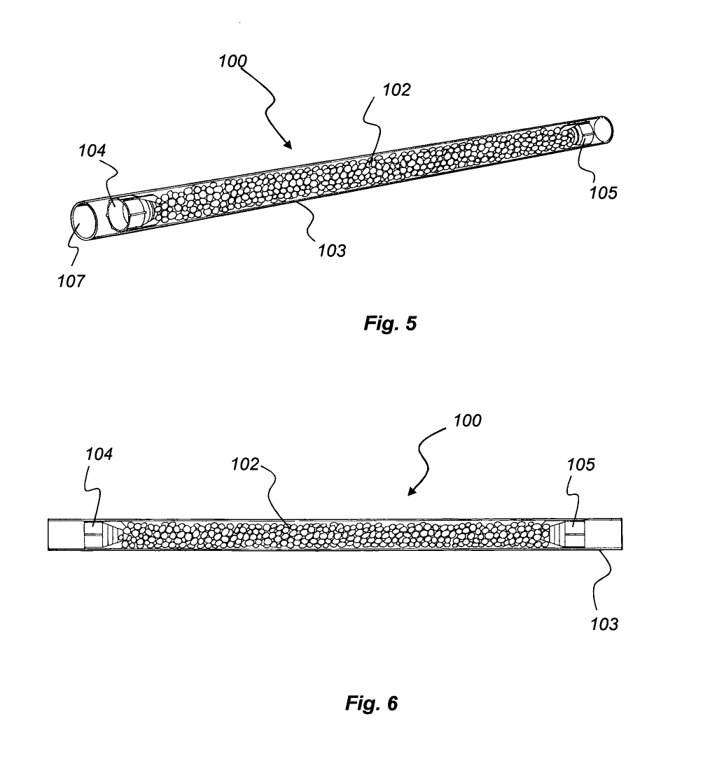

In another form, the beads may be used in purposely designed or adapted dispensing devices. One such device is shown in FIGS. 5 and 6 of the drawings which depict an apparatus including a receptacle 100 for containing a predetermined measure of the probiotic composition for dispersion.

The probiotic composition is in the form of discrete pellets 102 held within a body 103 of the receptacle 100. A pair of filters, 104 and 105, are disposed at opposite ends of the body, to retain the pellets, while permitting relatively unimpeded passage of the carrier liquid therethrough.

In the embodiment shown in FIGS. 5 and 6, the body 103 of the receptacle takes the form of a generally cylindrical tube including a solid sidewall 107 allowing its use as a straw. The filters 104 & 105 are adapted to form a pellet containment region within the receptacle retaining the pellets 102.

The filters shown in the figures are inserts formed separately and attached to the straw body. They include an attachment portion for engaging the straw body and a filtration section. However, the term “filter” as used herein is also intended to be interpreted broadly, as encompassing any form of porous or other barrier mechanism that functions to allow relatively unimpeded passage of a carrier liquid, while retaining the pellets or beads substantially within the receptacle, prior to dispersion or dissolution into the carrier liquid. Suitable filters may take a wide variety of forms including sieves, screens, grates, mesh materials, woven or non-woven fabrics, porous solids, granular beds, sponges, perforated plates, perforated or porous membranes, tortuous passageways, suitably dimensioned one-way or multi-way valves, and the like, or any combination of such forms, in any suitable shape or configuration, whether integrally formed, releasably connected or permanently secured in position. In particular, it should be understood that one or more filters may be integrally moulded or otherwise formed in conjunction with a side wall or other portion of the body of the receptacle and/or the straw.

In use, the carrier liquid is drawn through the receptacle by oral suction and the pellets 102 constituting the probiotic composition and optional additional ingredients are thereby progressively dissolved or otherwise dispersed into the carrier liquid within the straw, at the point of consumption.

While the filters are shown in the figures to be disposed at, on or adjacent opposite ends of the body, in other embodiments, one or both of the filters may be disposed at intermediate positions within or along the length of the body.

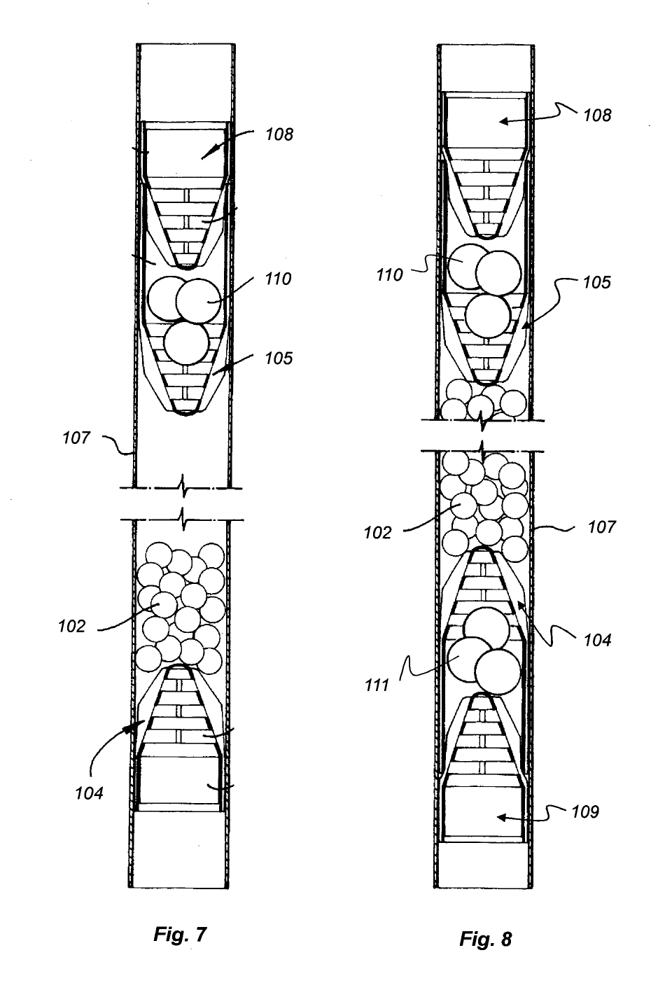

In other embodiments, as shown in FIGS. 7 and 8, one 108 or more 109 additional filters may be used to provide additional pellet containment regions. These additional pellet containment regions may be used to similarly contain one 110 or more 111 additional types of pellets in each respective chamber. Such a configuration is fully described in the applicant's previous application, WO 2008/055296 incorporated herein by reference. This also provides the receptacle as being formed as a separate and discrete component, adapted for connection to or integration with a straw as part of a subsequent process step, manufacturing operation or assembly procedure. This enables one of the types of beads or pellets to be inserted in the receptacle under controlled conditions corresponding to a pharmaceutical-grade production and packaging environment while another type of beads to be inserted into the straw under different, controlled conditions.

When applied to the straw as shown, the pellets or beads are generally spherical in shape, having an average diameter that is between 5% and 95% and preferably between 10% and 90%, of the internal diameter of the body. In other embodiments, the generally spherical pellets or beads are between 20% and 80%, and in other embodiments between 25% and around 75%, of the internal diameter of the body. The body itself, in some embodiments has an internal diameter of between 3 mm and 15 mm, and in some embodiments between 7 mm and 9 mm. In some embodiments, the pellets or beads have an average diameter of between 1 mm and 8 mm, and ideally between 1.5 mm and 5 mm. In cases of non-circular straws and/or bodies, or straws and or bodies of non-uniform cross section, it will be appreciated that the above dimensions form a rough guide. For instance, it is preferable that the maximum and minimum widths of such straws and/or bodies fall within the nominated 3 mm to 15 mm range and preferably within 5 mm to 12 mm.

In one particularly preferred embodiment the internal diameter of the straw is around 8 mm and the pellets are between 1 mm and 3 mm. In another particularly preferred embodiment the internal diameter of the straw is around 5 mm and the pellets are between 1 mm and 3 mm.

The probiotic beads or pellets can be used with an additional ingredient or ingredients within the straw, receptacle or tube whereby said probiotic microorganisms and said additional ingredient or ingredients are progressively dispersed into the carrier liquid within the straw, receptacle or tube. Additional ingredients can include pharmaceuticals, vitamins, minerals, nutritional supplements, health tonics, energy supplements, stimulants, colouring agents or flavouring agents may be used.

When using a layered pellet construction, the innermost layers of at least some of the pellets or beads contain and optionally such things as flavourings at concentrations greater than those in the outermost layers. Advantageously, this increase in concentration offsets the reduction in surface area of the pellets or beads as they progressively dissolve, thereby imparting a relatively uniform concentration of a probiotic microorganism and flavourings or other ingredients during consumption.

In some embodiments, the concentration and/or dissolution rate of the probiotics and optional additional ingredient(s)/component(s) are selected such that substantially all of them are dissolved and delivered upon consumption of a predetermined volume of an intended carrier liquid. In one such embodiment, the straw is packaged and sold in combination with a complementary container, which includes or is adapted to contain the predetermined volume of the intended carrier liquid.

In these and other respects, the invention represents a practical and commercially significant improvement over the prior art.

Although the invention has been described with reference to specific examples, it will be appreciated by those skilled in the art that the invention may be embodied in many other forms.

### Experimental Methodology

### 1. Probiotic Microorganisms

Suitable preparations of probiotic microorganisms in powder or liquid form for use in the present invention were obtained from different commercial sources: Danisco USA Inc. 3329 Agriculture Drive Madison, Wis., USA; Fonterra Centre, 9 Princes St, Auckland 1010 New Zealand; P.L. Thomas & Co. Inc. 119 Headquarters Plaza, Morristown, N.J. 07960 USA; Lallemand SAS, 19 rue des Briquetiers, BP 59, 31702 Blagnac Cedex, France; Want Want Holdings Limited. No. 1088, east Hong Song Road, Shanghai 201103, China; DSM, Alexander Fleminglaan 1, 2613 AX Delft, The Netherlands; Probi AB, Solvegatan 41, SE-22370, Lund, Sweden; Blis Technologies Limited, Level 1, Centre of Innovation, St David st, Dunedin, 9016, New Zealand; ProBiOz Pty Ltd 22 Goorawahl Avenue, La Perouse NSW 2036, Australia.

If desired, probiotic microorganisms may be obtained as live cultures from sources such as ATCC, inoculated and cultured in standard culture media, harvested and freeze-dried (lyophilized), techniques all well known to those skilled in the art, to obtain powdered preparations of probiotic microorganisms suitable for use in the present invention.

### 2. Determination of Probiotic Viability

For enumeration of the probiotic in bulk powder, the spread plate technique was used. In brief, a ten-fold dilution of the powder containing a probiotic microorganism was made in sterile peptone water with added Tween-80 and the sample was blended for one minute. After gentle mixing for 30 minutes, the sample was again blended prior to ten-fold serial dilution. From appropriate dilutions, 100 microliter aliquots were inoculated onto agar, for example MRS agar (for Lactobacillus) or RCA (for Bifidobacterium and other species). Plates were incubated anaerobically for 48-72 hours. For each sample, the assay was carried out in triplicate (triplicate dilution series each generating triplicate spread plates). For enumeration of viable probiotics in beads during development and stability studies, samples were similarly 10-fold serially diluted but instead of spread plates using 100 microliter inocula, the drop plate method using 10 microliter drops were used.

### 3. Stability Studies

The stability of the probiotic in the beads was assessed by storage at ambient/room temperatures (22-25° C., 25° C. or 35° C.). The beads were stored for the stability study either:
(a) in laminated film sachets or in plastic screw capped plastic bottles in a desiccator with silica gel;
(b) in plastic sample jars with screw caps and no additional protection from ambient conditions; or
(c) in 8 mm straws individually wrapped and heat sealed in laminated metalised or foil based film laminates and stored at 25° C. or 35° C. with 60% relative humidity.

### EXAMPLES

### Example 1

### Probiotic Coating of Beads Using Sugar Syrup, PVP or PVA

This process involves coating beads using a sugar crystal as the bead core (seed) and panning the bead by successive additions of tapioca starch-maltodextrin powder and sugar syrup. Beads were warmed in a pan prior to the addition of a quantity of sugar syrup immediately followed by a quantity of powder and then syrup and then powder. The process is successively repeated until the bead is the desired size, preferably 1.9-2 mm. The beads were coated with the probiotic using the panning process to produce layered beads. The probiotic was added to the tapioca starch-maltodextrin powder. The process was also modified to include skim milk in the tapioca-maltodextrin powder (10%) and using polyvinylpovione (PVP) adhesive or polyvinylalcohol (PVA) adhesive instead of sugar syrup.

To provide 2 kg of layered 2 mm beads, five to six coats were applied to the beads in successive panning steps using 250 g of powder mix and 95 ml of sugar syrup or PVP or PVA. Beads were produced using either sugar syrup or PVP or PVA in the final coats with the probiotic. The coated beads were then freeze dried in which form they could be stored while substantially maintaining the viability of the probiotic microorganisms.

Results—Process 1: Survival of Lactobacillus acidophilus UN 08 Using Method of Inclusion of Probiotic in the Matrix

| Added bacteria cfu/g | Bacteria in finished beads cfu/g |
|---|---|
| 1.3 × 10⁸ | 2.3 × 10⁷ |

### Example 2

### Probiotic Coating of Beads Using Adhesive Syrup (Addition of Probiotic to Powder)

This process involves coating beads using a sugar crystal as the bead core (seed) and panning the bead by successive additions of tapioca starch-maltodextrin powder and sugar syrup. The beads were coated with the probiotic by the panning process as used to produce layered beads of approximately 2 mm in diameter. The probiotic was added to a tapioca starch-maltodextrin powder with the following composition to yield a probiotic concentration in the finished beads in the range 10⁶ to 10¹⁰ viable cells per gram as required for the particular application:

| | |
|---|---|
| Tapioca starch | 80 g |
| Maltodextrin | 20 g |
| Silica dioxide | 2.5 g |
| Sucralose | 1.0 g (optional ingredient) |

When coating the beads with the probiotic powder, the sugar syrup was replaced with an adhesive syrup. The composition of the adhesive syrup was as follows:

| | |
|---|---|
| Gum acacia | 2.5% |
| Soy protein | 5% |
| Canola oil | 2% |
| Lecithin | 0.25% |
| Lactose | 10% |
| Maltodextrin | 30% |
| Water | 50% |

Five to six coats were applied to the beads in successive panning steps using 275 g of powder mix and 100 ml of adhesive syrup to result in 2 kg of layered 2 mm beads. Panning temperature was 31° C. The layered beads were freeze dried in which form they could be stored while substantially maintaining the viability of the probiotic microorganisms.

Results—Survival of Lactobacillus fermentum UN 06 During and on Storage at 25° C. When Produced Using Process.

| Strain | Added cells | Finished product (T₀) | 4 weeks | 8 weeks |
|---|---|---|---|---|
| UN 06 | 4.7 × 10⁵ | 6.7 × 10⁵ | 1.5 × 10⁵ | 2.8 × 10⁵ |

Survival of Lactobacillus fermentum UN 06 during and on storage at 35° C.

| Strain | Added cells | Finished product (T₀) | 4 weeks |
|---|---|---|---|
| UN 06 | 4.7 × 10⁵ | 6.7 × 10⁵ | 9.2 × 10⁴ |

A variation of the above process is to add the probiotic to the syrup instead of to the powder.

### Example 3

### Probiotic Coating of Beads Using Sucrose-Free Syrup and an Extra Coating Over the Probiotic

This process involves producing beads using a sugar crystal bead core as per Example 2, panning the bead by successive additions of tapioca starch-maltodextrin powder and sugar syrup to produce beads including the probiotic using the panning process as used to produce layered beads of approximately 2 mm in diameter prepared in accordance with example 1. The probiotic was added to a tapioca starch-maltodextrin powder with the following composition and to yield a probiotic concentration in the finished beads in the range of 10⁶ to 10¹⁰ viable cells per gram as required for the particular application. The composition of the tapioca starch-maltodextrin powder was as follows:

| | | |
|---|---|---|
| Tapioca starch | 80 | g |
| Maltodextrin | 20 | g |
| Silica dioxide | 1.0 | g |
| Sucralose | 2.5-5.0 | g |

(optional ingredient)

When coating the beads with the probiotic powder an adhesive syrup was used. The composition of the syrup was as follows:

| | |
|---|---|
| Gum acacia | 1.25% |
| Soy protein | 5% |
| Canola oil | 1% |
| Lecithin | 0.25% |
| Lactose | 10% |
| Maltodextrin | 30% |
| Water | 50% |

Five to six coats of material that included the probiotic was applied to the beads in successive panning steps using a total of approximately 275 g of powder mix and 100 mls of syrup to result in 2-3 kg of layered 2 mm beads. The panning temperature was 30-33° C. After addition of the 5-6 probiotic layers, an additional 5 layers of tapioca-maltodextrin mix was added without any probiotic. When applying the subsequent layers that did not include probiotic the sugar syrup was used.

Accordingly, in this example the layered beads included the probiotic in the inner layers and not in the outer layers. The layered beads were freeze dried in which form they could be stored while substantially maintaining the viability of the probiotic microorganisms.

### Results: Use of a Range of Strains of Probiotic Bacteria-Showing Viable Cells at Production (‘E’ Refers to Exponential)

| Bacteria | Added cells | Viable cells in beads |
|---|---|---|
| Streptococcus thermophilus | 1.5E+8 | 8.9E+7 |
| Lactobacillus bulgaricus | 1.0E+4 | 1.1E+4 |
| Bacillus coagulans | 1.1E+6 | 7.5E+5 |
| Lactobacillus fermentum | 8.3E+4 | 4.8E+4 |
| Bifidobacterium infantis | 1.8E+8 | 1.8E+7 |
| Lactobacillus rhamnosus | 1.4E+8 | 6.1E+7 |
| Lactobacillus plantarum | 3.4E+8 | 5.9E+7 |
| Lactobacillus acidophilus | 7.5E+6 | 3.2E+6 |

### Example 4

### Probiotic Coating of Beads in Non-Aqueous Binding Systems

This process involves coating beads using canola oil and using the bead core (seed), and coating the bead by successive additions of tapioca starch-maltodextrin powder and oil alone or with added gum acacia (2.5%) or silicon dioxide. Each successive layer applied as a mixture of the 3 ingredients. The beads were coated with the probiotic by the panning process as used to produce layered beads of approximately 2 mm in diameter (as described in Example 2). The probiotic was added to a tapioca starch-maltodextrin powder with the following composition to yield a probiotic concentration in the finished beads in the range log 6 to log 10 as required for the particular application:

| | |
|---|---|
| Tapioca starch | 80 g |
| Maltodextrin | 20 g |
| Silica dioxide | 2.5 g |

Five to six coats were applied to the beads in successive panning steps using a total of approximately 275 g of powder mix and 100 ml of oil to result in 2-3 kg of layered 2 mm beads. Panning temperature was ambient. After addition of the 5-6 probiotic layers, an additional 5 layers of tapioca-maltodextrin mix without added probiotic was added. The layered beads were either used directly or freeze dried to yield a water activity of 0.2 or less.

### Results—Survival of Probiotics Using Oil as the Agent to Bind the Probiotic Containing Powder to the Beads.

Result show the added number of viable probiotic Bifidobacterium infantis cells and the number of viable cells in beads after production, expressed as colony forming units per gram of beads (enumerated as outlined in point 1 under experimental methodology:

| Added probiotic | Probiotic in beads |
|---|---|
| 2.4 × 10⁹ | 3.0 × 10⁸ |

### Example 5

### Probiotic Impregnated/Coated Beads Using Hydrophobic Liquid

A dried core bead prepared as described in Example 1 (composed of 48% sugar, 40% starch, 10% maltodextrin and 2% gum) was coated with a canola oil suspension carrying the probiotic in the form of a freeze dried powder.

The core bead is manufactured in a coating pan using the standard methodology of sugar coating (as described in Example 1). The core bead is dried to remove moisture, preferably to a level of 0.2% or less, The type of drying process used is unimportant and may be undertaken through oven drying, freeze drying, vacuum drying or similar to achieve the dry, porous bead. Advantageously the drying process also makes the core bead porous. The probiotic mix applied is a suspension of probiotic powder (35%) in canola oil (65%). The probiotic suspension may include a low level (<2%) of antioxidant such as tocopherol or Rosemary oficianalis extract. The oil suspension may be applied by spraying, pouring or showering it onto the core beads. A proportion of the applied oil suspension may impregnate the porous core bead. The bead mass is then tumbled using a coating pan, mixing vessel or other means to ensure even dispersion of the oil suspension over the core beads. Tumbling continues for 10 to 20 minutes.

### Results—Survival of Probiotics Using Oil as the Agent to Bind the Probiotic Containing Powder to the Beads.

Result detailed in the tables below show the added number of viable probiotic Bifidobacterium lactis, Lactobacillus acidophilus or Streptococcus salivarus cells and the number of viable cells in beads after production as well as at time points of 3 months and 6 months where available, expressed as colony forming units per gram of beads (enumerated as outlined in point 1 under experimental methodology):

Results—Bifidobacterium lactis:

| Added probiotic | Probiotic in beads | 3 Month Count | 6 Month count |
|---|---|---|---|
| 8.6 × 10⁹ | 8.7 × 10⁹ | 6.6 × 10⁹ | 1.6 × 10¹⁰ |
| 9.6 × 10⁹ | 7.8 × 10⁹ | 7.1 × 10⁹ | 4.1 × 10¹⁰ |
| 9.6 × 10⁹ | 1.6 × 10¹⁰ | 6.4 × 10⁹ | 1.1 × 10¹⁰ |
| 9.6 × 10⁹ | 6.3 × 10⁹ | 1.2 × 10⁹ | 1.6 × 10⁹ |
| 9.6 × 10⁹ | 4.1 × 10⁹ | 1.2 × 10⁹ | 3.1 × 10⁹ |
| 9.6 × 10⁹ | 6.3 × 10⁹ | 2.8 × 10⁹ | 8.2 × 10⁹ |

Results—Lactobacillus acidophilus:

| Added probiotic | Probiotic in beads | 3 Month Count |
|---|---|---|
| 5.4 × 10⁹ | 7.6 × 10⁹ | 4.5 × 10⁹ |
| 5.4 × 10⁹ | 5.1 × 10⁹ | 4.1 × 10⁹ |
| 5.4 × 10⁹ | 5.0 × 10⁹ | 3.0 × 10⁹ |
| 5.4 × 10⁹ | 5.6 × 10⁹ | 7.8 × 10⁹ |
| 5.4 × 10⁹ | 5.4 × 10⁹ | 5.5 × 10⁹ |
| 5.4 × 10⁹ | 3.9 × 10⁹ | 5.7 × 10⁹ |

Results—Streptococcus salivarius:

| Added probiotic | Probiotic in beads | 3 Month Count |
|---|---|---|
| 1.8 × 10⁹ | 5.7 × 10⁸ | 2.6 × 10⁸ |
| 1.8 × 10⁹ | 5.7 × 10⁸ | 3.7 × 10⁸ |
| 1.8 × 10⁹ | 3.5 × 10⁹ | 6.4 × 10⁹ |
| 1.8 × 10⁹ | 4.8 × 10⁹ | |

### Example 6

### Probiotic Impregnated Beads with a Protective Coating

A dried core bead (composed of 40% sugar, 48%starch and 12% maltodextrin) was coated with canola oil suspension carrying the probiotic used as a freeze dried powder. Saturated beads were then coated with a protective layer of carbohydrate in this case, dextrous monohydrate.

The core bead is dried to make it porous. The type of drying process used is unimportant and may be undertaken through oven drying, freeze drying, vacuum drying or similar to achieve the dry, porous bead. The probiotic mix applied is a suspension of probiotic powder (35%) in canola oil (65%). The oil suspension may be applied by spraying, pouring or showering it onto the core beads

After freezing overnight (at −18° C.), the beads were then coated with a mixture of dextrous monohydrate (85%) and water (15%) heated to 75° C. along with some dry dextrous monohydrate (100%).

### Results—Survival of Probiotics Using Oil as the Agent to Bind the Probiotic Containing Powder to the Beads.

Result detailed in the table below show the added number of viable probiotic Lactobacillus rhamnosus cells and the number of viable cells in beads after production, expressed as colony forming units per gram of beads (enumerated as outlined in point 1 under experimental methodology):

| | Added probiotic | Probiotic in beads |
|---|---|---|
| Results without coating | 3.07 × 10⁹ | 2.76 × 10⁹ |
| Results with coating | 2.61 × 10⁹ | 2.5 × 10⁸ |

### Example 7

### Probiotic-Coated Beads Using Calcium Carbonate and Oil

The core bead used in this process may be a sugar seed or a layered core bead. The core beads may be prepared as described in Example 2, and may be further treated (eg. dried) as described in Example 5.

Canola oil (or any suitable food-grade oil) is mixed with the probiotic bacteria 40% culture and 60% oil and applied to the core bead. The mixture can be applied by a process as described in Example 5. The probiotic-containing beads are then coated with calcium carbonate 100% calcium carbonate powder applied in a panning process adhering to the oil coated bead. The calcium carbonate interacts with the oil to form a paste-like layer which protects the probiotic bacteria by sequestering the oil in the vicinity of the bacteria and excluding water. The use of calcium carbonate is not critical to this process. Any mineral salt that is capable of forming a paste-like composition with an oil may be used.

If desired several layers of probiotic-oil mix/calcium carbonate may be applied to the core bead to increase the probiotic loading of the beads. Each layer is completed by adding enough dry calcium carbonate powder to ensure most of the oil is used in forming the paste. Following this another application of oil then more calcium carbonate powder.

Alternatively, the probiotic bacteria may be mixed with oil and calcium carbonate and this mixture applied to the core bead, in multiple coatings if desired, and finally coated with a calcium carbonate protective layer.

In yet another alternative process, oil may be mixed with a food-grade polyol such as propylene glycol before adding the probiotic bacteria. This mixture can then be applied to the core beads as described above or mixed with calcium carbonate before application to the core beads.

An anti-oxidant such as ascorbic acid, tocopherol, rosemary extract or other may be added to the oil to prevent its oxidation and breakdown on storage. Colouring and/or flavours may also be added to the oil, as can functional ingredients such as, for example, omega-3 oil, vitamins and the like

A disintegrant, such as ascorbic acid or starch or many available in the pharmaceutical arena may be added to the calcium carbonate layer to assist in release of the probiotic bacteria when the probiotic beads come into contact with a fluid.

1. A probiotic composition comprising a probiotic microorganism embedded within a matrix, said matrix substantially maintaining the viability of said microorganisms, whereby said matrix releases said microorganisms into and upon contact with a liquid carrier.

2. A probiotic composition according to claim 1, wherein said composition is provided in a form selected from the group consisting of particulates, powders, pellets, ribbons, blocks, cubes, polygons, spheres, discs, tablets, and cylinders/rods.

3. A probiotic composition in pellet form comprising a probiotic microorganism embedded within a matrix, said matrix substantially maintaining the viability of said microorganisms, whereby said matrix releases said microorganisms into and upon contact with a liquid carrier.

4. A probiotic composition in pellet form comprising a core bead and a probiotic microorganism embedded within a matrix, said matrix substantially maintaining the viability of said microorganisms, wherein the matrix is disposed on or in the core bead and whereby said matrix releases said microorganisms into and upon contact with a liquid carrier.

5. A probiotic composition in pellet form comprising a core bead and a probiotic microorganism embedded within a matrix, said matrix substantially maintaining the viability of said microorganisms, wherein the matrix is disposed on the core bead in plurality of layers to form a layered pellet and whereby said matrix releases said microorganisms into and upon contact with a liquid carrier.

6. A probiotic composition according to any one of claims 1 to 5, wherein said matrix includes one or more components selected from the group consisting of carbohydrates, milk products, binding agents, thickening agents, emulsifiers, oils, fats, fatty acids, water, silicon dioxide, vegetable or animal proteins, resistant starches, dextrins, sugars, sucralose, gum acacia, and lecithin.

7. A probiotic composition according to any one of claims 1 to 6, wherein said probiotic microorganism is a bacterium or a yeast.

8. A probiotic composition according to claim 7, wherein said probiotic microorganism is selected from the group comprising genera Lactobacillus, Bifidobacterium, Lactococcus, Propionibacterium, Bacillus, Enterococcus, Escherichia, Streptococcus and Saccharomyces.

9. A probiotic composition according to claim 7, wherein said probiotic microorganism is selected from the group comprising genera Lactobacillus, Streptococcus and Bifidobacterium.

10. A probiotic composition according to claim 6, wherein said probiotic microorganism is selected from the group comprising Lactobacillus acidophilus, Lactobacillus rhamnosus, Lactobacillus fermentum, Lactobacillus casei, Lactobacillus bulgaricus, Lactobacillus gasseri, Lactobacillus helveticus, Lactobacillus johnsonii, Lactobacillus lactis, Lactobacillus plantarum, Lactobacillus reuteri, Lactobacillus salivarius, Lactobacillus paracasei, Bifidobacterium sp, Bifidobacterium longum, Bifidobacterium infantis, Bifidobacterium animalis, Bifidobacterium bifidum, Bifidobacterium adelocentis, Bifidobacterium lactis, Enterococcus faecalis, Enterococcus faecium, Lactococcus lactis, Streptococcus salivarius, Saccharomyces cereviscae and Saccharomyces boulardii.

11. A probiotic composition according to any one of claims 1 to 10, wherein the matrix is soluble in the liquid carrier.

12. A probiotic composition according to any one of claims 1 to 10, wherein the matrix is dispersed or eroded by the liquid carrier.

13. A probiotic composition according to any one of claims 1 to 10, wherein the matrix is non-aqueous.

14. A probiotic composition according to claim 13, wherein the matrix is an oil.

15. A probiotic composition according to claim 14, wherein the oil is selected from olive oil, palm oil, soybean oil, canola oil, pumpkin seed oil, corn oil, sunflower oil, safflower oil, peanut oil, grape seed oil, sesame oil, argan oil, coconut oil and rice bran oil.

16. A probiotic composition according to any one of claims 4 to 15, wherein the core bead is porous or semi-porous.

17. A probiotic composition according to claim 16, wherein the core bead is soluble in the liquid carrier.

18. A probiotic composition according to claim 16, wherein the core bead is dispersed or eroded by the liquid carrier.

19. A probiotic composition according to any one of claims 4 to 13, wherein the probiotic microorganism is disposed in at least one matrix layer.

20. A probiotic composition according to any one of claims 5 to 19, wherein the probiotic microorganism is disposed in more than one matrix layer and wherein each matrix layer includes a different probiotic microorganism.

21. A probiotic composition according to any one of claims 1 to 20, wherein the matrix and/or the core bead further comprise additives selected from the group consisting of vitamins, minerals, nutritional supplements, health tonics, colouring agents, flavouring agents and pharmaceutical agents.

22. A probiotic composition according to any one of claims 1 to 20 for use in a container or receptacle.

23. A probiotic composition according to claim 22 wherein the container or the receptacle is a sachet or a drinking straw.

24. A probiotic composition according to any one of claims 2 to 23, wherein the pellets have a size range of about 1 mm to about 3 mm.

25. A probiotic composition according to any one of claims 1 to 24, wherein the moisture content of the matrix and/or the bead core is about 0.2% or less.

26. A method of producing a layered pellet comprising a probiotic microorganism said method comprising:
i) providing a bead core;
ii) adhering a layer of matrix material to said core to provide a layered pellet; and
iii) optionally further contacting said layered pellet one or more times with matrix material to apply one or more additional layers,
wherein at least one of steps i, ii or iii includes a matrix material comprising a probiotic microorganism, such that the layered pellet includes a probiotic microorganism.

27. A method of producing a probiotic composition in pellet form, said method comprising:
i) providing a porous or semi-porous core bead;
ii) impregnating said core with a matrix comprising a probiotic microorganism; and
iii) optionally applying one or more surface coating layers on said impregnated core.

28. A pellet composition comprising a probiotic microorganism when produced by a method according to claim 26 or claim 27.

29. A receptacle containing a probiotic composition according to any one of claims 2 to 25 or 28, said receptacle including a body adapted to contain said plurality of pellets or beads, and a pair of filters disposed in spaced apart relationship and adapted substantially to retain said plurality of pellets or beads within the body prior to dissolution while permitting relatively unimpeded passage of a carrier liquid therethrough.

30. A receptacle according to claim 29, said receptacle being a part of, or adapted for connection to, an elongate tube adapted for use as a straw, thereby enabling the carrier liquid to pass through the receptacle and the probiotic microorganisms to be dispersed into the carrier liquid.

31. A receptacle according to claim 30 wherein said tube has a generally circular cross section, the internal diameter of said tube being between 3 mm and 15 mm.

32. A receptacle according to claim 31 wherein the internal diameter of said tube is around 8 mm.

33. A receptacle according to claim 31 wherein the internal diameter of said tube is around 5 mm.

34. A receptacle according to any one of claims 29 to 33 wherein said straw includes a measure of probiotic composition having a predetermined dose of probiotic.

35. A receptacle according to any one of claims 29 to 34 wherein said straw is hermetically sealed in a wrapper.

36. A container containing a probiotic composition according to any one of claims 1 to 25 or 28, wherein said container is hermetically sealed.

37. A container according to claim 36, wherein said container is a disposable sachet.

38. A container according to claim 37, wherein said container includes a measure of probiotic composition having a predetermined dose of probiotic.
